(19) United States
(12) Patent Application Publication
(10) Pub. No.: US 2004/0102606 A1
(43) Pub. Date: May 27, 2004

(54) Histone H2A -derived peptides useful in gene delivery

(76) Inventors: Danuta Balicki, Montreal (CA); Ernest Beutler, La Jolla, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(21) Appl. No.: 10/258,637

(22) PCT Filed: Apr. 24, 2001
(86) PCT No.: PCT/EP01/04621

(51) Int. Cl. A61K 48/00; C07K 9/00

(52) U.S. Cl. 530/322; 514/8; 514/44

(57) ABSTRACT

The present invention provides a novel gene delivery system in which a gene delivery facilitating peptide, generally derived from Histone H2A, is complexed with a nucleic acid for efficient and stable delivery of the nucleic acid into a cell, ultimately to the nucleus. Such peptide-mediated gene delivery is based on the principal that unneutralized positive charges on the histone are bound electrostatically both by the negatively charged phosphate backbone of DNA and that nuclear targeting signals in histones improve trafficking of the DNA to the nucleus for transcription.

### TECHNICAL FIELD

The invention relates to peptides derived from histone H2A, hereinafter referred to as H2A, and use thereof in facilitating gene delivery of a nucleic acid into a cell More specifically, the invention describes H2A-derived peptide-nucleic acid complexes useful in transfecting cells in vitro and in vivo, and obtaining nuclear localization of the nucleic acid. The invention further relates to H2A peptide-nucleic acid complexes in which the nucleic acid is an expression vector further comprising a nucleotide sequence encoding at least one H2A peptide having a nuclear localization signal sequence. Methods of making and using the H2A peptide-nucleic acid complexes and expression vectors for enhancing gene delivery, particularly cellular transfection and nuclear localization, are described. Articles of manufacture are also described containing H2A peptides and packaging material, the latter including a label for indicating the use of H2A peptides in facilitating gene delivery.

### BACKGROUND

Expressing exogenous nucleic acids in cells, both in vitro and in vivo, allows for a variety of applications including the investigation of cellular regulation, production of large amounts of recombinant protein, cloning of genes, replacement of defective or absent genes, or inhibition or activation of cellular regulation.

Both viral and non-viral methods have been developed to deliver or transfer molecules into cells. Viral and non-viral approaches for use in gene therapy have been the subject of extensive reviews regarding their respective including advantages and disadvantages (for example, see review articles, Romano et al, Stem Cells, 18:19-39, 2000; Mountain, Trends in Biotech., 18:119-128, 2000; Clackson, Gene Ther., 7:120-125, 2000; and Mahato, J. Drug Targeting, 7:249-268, 1999).

One of the main challenges of gene therapy is successful nucleic acid delivery to the cell nucleus with minimal levels of toxicity to the host. Gene therapy can potentially correct genetic disease, through the replacement of a deficient enzyme (for example, see Rolland, Critical Reviews in Therapeutic Drug Carrier Systems, 15:143-198, 1998). Its methods are intended to overcome some limitations associated with the clinical use of protein drugs, including high cost of manufacture, low bioavailability, and poor pharmacokinetics (Stewart et al., Hum. Gene Ther., 3:267-275, 1992). Examples of target genetic diseases include Gaucher disease (for example, see Balicki and Beutler, Medicine (Baltimore), 74:305-323, 1995), adenosine deaminase deficiency (for example, see Dunbar et al., Hum Gene Ther., 10:477-488 1999), and cystic fibrosis (for example, see Crystal et al., Nat. Genet., 8:42-51, 1994). It also has therapeutic applications in the treatment of acquired diseases such as cancer, AIDS, arthritis, and cardiovascular disease and the like.

For a gene therapy modality to be successful, a useful strategy is to deliver the target gene to the nucleus for it to be transcribed and translated. In this strategy, the first barrier to overcome is the cell membrane. Then it must be protected from nucleases in the cytoplasm and overcome the possibility of endosomal entrapment. Finally, the nucleic acid must enter the nucleus where the target gene can proceed to be transcribed, translated, and then the daughter protein trafficks to the cellular location where it has a function. The ideal gene delivery system is non-toxic, non-immunogenic, easy to produce in large quantities, and it is efficient in protecting and delivering DNA into cells, preferably with a specificity toward a particular cell type.

Since viruses have evolved to perform this function as efficiently as possible, the main focus of this type of therapeutic effort has been the use of modified viral vectors. However, the imitations of viral vectors have included potential pathogenicity and antigenicity, and attention has therefore turned to the promise associated with non-viral means of delivering genes.

The key to successful non-viral gene delivery is the careful construction of the transfecting complex. This includes the incorporation of beneficial aspects of viral gene delivery, such as DNA condensation. Histones are potentially good building blocks in the formation of effective transfecting complexes because of their DNA-condensing capacities. In addition, the unneutralized positive charges of histones could be bound electrostatically by the negatively charged phosphate backbone of DNA, and nuclear targeting signals in histones might improve trafficking of the DNA to the nucleus where it could be transcribed. The efficacy of histones in DNA transfection has been described (Balicki and Beutler, Mol. Med., 3:782-787, 1997; Budker et al., Biotechniques, 23:139-147, 1997; Chen et al., Hum Gene Ther., 5:429-435, 1994; Fritz et al., Hum. Gene Ther., 7:1395-1404, 1996; Hagstrom et al., Biochim. Biophys. Acta, 1284:47-55, 1996). Histone H2A was by far the most efficient of all histone subclasses in mediating DNA transfection (Balicki and Beutler, supra, 1997).

These same persons, the present inventors, have now discovered that the entire H2A sequence is not essential for mediating efficient delivery of nucleic acids into cells. They have identified that a short fragment of H2A molecule is responsible for the biological function. They have further discovered that various peptide substitutions of the H2A fragment are also efficient at mediating gene delivery, including transfection and nuclear localization capability. Thus, the present invention now provides an improved efficient gene delivery system only requiring the formation of a complex of a short peptide derived from H2A with a nucleic acid in a delivery enhancing medium that overcomes the limitations of current gene delivery approaches including viral and non-viral means.

### BRIEF DESCRIPTION OF THE FIGURES

FIG. 1 shows the transfection activity of peptides at an equimolar ratio to the peak conditions for transfection of peptide 1, as well as at a peptide concentration two-fold greater (2× molar) and two-fold less (1/2×molar). The results are the average β-galactosidase production +/− the standard error of the mean.

FIG. 2 summarizes the transfection activity of 17-mers that are identical to P1 except for certain substitutions. Peptide P11 is a short stretch of amino acids found in the N-terminus of Peptide 1. Glycines or arginines are substituted in the remaining peptides. An additional arginine was also added at either end of peptide P1. Some increased transfection activity occurs when the N-terminal arginine of peptide P1 is substituted by serine, possibly by freeing this end.

### SUMMARY OF THE INVENTION

In one aspect the present invention provides an isolated gene delivery facilitating peptide comprising at least 7 amino acids, preferredly 17 amino acids, derived from the N-terminal region of histone H2A, wherein the peptide exhibits transfection activity and nuclear localization activity. Also provided is a complex comprising such a peptide of the invention complexed with a nucleic acid. Further, a solution comprising the complex of the invention and a transfection enhancing medium is provided. Furthermore, a nucleic acid coding for a peptide of the invention is provided.

In another aspect a pharmaceutical composition comprising a transfection enhancing amount of a complex according to the invention in a pharmaceutically acceptable carrier is provided.

In a further aspect there is provided a method of preparing a complex comprising mixing a peptide according to the invention with a nucleic acid in a transfection enhancing medium to form a peptide nucleic acid complex. Also provided is a method of transfecting a cell comprising administering a complex according to the invention to the cell. Accordingly, a cell transfected according to the method of the invention is also provided.

In yet another aspect there is provided an article of manufacture comprising a packaging material and contained therein in a separate container a gene delivery facilitating H2A-derived peptide according to the invention, wherein the peptide is effective for delivering a nucleic acid into a cell, and wherein the packaging material comprises a label which indicates that the peptide can be used for delivering a nucleic acid into a cell when a H2A-derived peptide nucleic acid complex is formed.

Also provided is an article of manufacture comprising a packaging material and contained therein in a separate container a pharmaceutical composition comprising a gene delivery facilitating H2A-derived peptide according to the invention, in a pharmaceutically acceptable carrier, wherein the peptide is effective for delivering a nucleic acid into a cell, and wherein the packaging material comprises a label which indicates that the peptide can be used for delivering a nucleic acid into a cell when a H2A-derived peptide nucleic acid complex is formed.

### DETAILED DESCRIPTION OF THE INVENTION

The present invention now provides a novel gene delivery system in which a gene delivery facilitating peptide, generally derived from Histone H2A, is complexed with a nucleic acid for efficient and stable delivery of the nucleic acid into a cell, ultimately to the nucleus. Peptide-mediated gene delivery is based on the principal that unneutralized positive charges on the histone are bound electrostatically both by the negatively charged phosphate backbone of DNA and that nuclear targeting signals in histones improve trafficking of the DNA to the nucleus for transcription.

In eukaryotic cells, the highly conserved histones assemble into a nucleosome core consisting of two molecules each of histone H2A, H2B, H3, and H4. This octamer wraps a 146 base pair stretch of DNA. In the higher order histone-DNA assemblies termed chromatin, a fifth histone, H1, associates with DNA linkers between core nucleosomes. Together the five histones are essential for packaging genomic DNA.

Calf thymus histone H2A is identical to human histone H2A. They are both 129 amino acid basic proteins with the following sequence: SGRGKQGGKARAKAKTRSSRAGLQFPVGRVHRLLRKGNYAERVGAGAPVYLAA VLEYLTAEILELAGNAARDNKKTRIIPRHLQLAIRNDEELNKLLGKVTIAQGGVLP NIQAVLLPKKTESHHKAKGK. The gene and encoded amino acid sequence for human H2A are available on GenBank with Accession Number Z83742.

The motif structure of histone H2A reveals the presence of 2 right-handed 310 helices encompassing amino acids 5-7 and 113-115. Histone H2A also is composed of 5 right-handed alpha helices between amino acids 17-21, 27-35, 45-72, 80-88, and 91-96. The transfection activity of histone H2A is specific to this molecule and not merely due to the presence of positive charge (Balicki and Beutler, supra, 1997). Transfections of COS7 cells (African green monkey SV40-transformed kidney cells) with a β-galactosidase reporter plasmid using poly-L-lysine, poly-L-arginine, and a mixture of poly-L-lysine and poly-L-arginine in equimolar ratios to their representation in histone H2A were ineffective. Transfections with cationic molecules such as polybrene, spermine, and spermidine were also ineffective. Interestingly, all of the cationic polymers tested bound to DNA, as demonstrated by agarose gel electrophoresis. These results suggest that DNA binding alone is insufficient for transfection, and that there is something particular to the sequence of amino acids in histone H2A that is responsible for its remarkable capacity to efficiently mediate gene delivery. Singh and Rigby (Nuc. Acids Res., 24:3113-3114, 1996) reported that efficiency of retroviral gene transfer increases in the presence of histone H2A.

The ability of histone H2A to mediate gene transfection more efficiently than positively charged molecules and other histones suggests that the unique components of the histone H2A-DNA interaction are key to efficient gene uptake. In the present invention, the dual role of histone H2A in DNA-binding and nuclear localization provides for transfection-competent protein and peptide constructs to improve the efficiency of the prior approaches.

In order to probe the structural role of histone H2A and the active peptide corresponding to the amino or N-terminal 36 residues, the published crystal structure of the nucleosome was analyzed using the program Xfit (McRee, J. Struct. Biol., 125:156-165, 1999) and custom software developed by that group. Overall, DNA interactions across the core histones are fairly widely distributed between proteins and along the protein sequences such that few contiguous peptide fragments contribute substantially to DNA binding. Histone H2A, however, exhibits a number of features in the nucleosome-DNA complex that may explain its role in transfection.

The first structural feature of histone H2A likely to be important for transfection is the N-terminal end of histone H2A, which interacts with DNA in a fashion analogous to a “clip”. Structurally, this clip is formed from two short e-helices (17-22 and 26-36) preceded by an extended and poorly ordered N-terminal extension (4-16) and interrupted by a short loop (22-25) that allows the two α-helices of the clip to anchor three adjacent phosphates on one strand of DNA. This clip positions the positively charged N-terminus along the DNA minor groove for four base pairs of 3′ phosphates bound by the clip, although the disorder of the N-terminus suggests that these interactions are somewhat weak. Together, the N-terminal 36 amino acids of H2A constitute one of the larger contiguous interaction surfaces in the core nucleosome.

The second feature of histone H2A that potentially defines transfection activity of the full-length protein and the derived peptides are the fourteen N-terminal amino acids. Although this region is poorly ordered in the crystal structure, it has been conserved between species. This positively charged region can function as a nuclear localization signal (NLS) which have been identified for histones H1 but not H2A. Thus, in the present invention, the active portion of H2A in facilitating transfection and nuclear localization spanning residues 1-36 and that possesses both the NLS and the DNA clip has been identified.

A. Gene Delivery Facilitating H2A-Derived Peptides

In one aspect, the invention provides a peptide derived from H2A that functions in nucleic acid delivery inside a cell. In particular, the peptides of the invention are useful in mediating entry of a nucleic acid in complex therewith into the cell as well as entry into the nucleus. As a result, efficient gene delivery transfer into the nucleus of the cell is achieved with the compositions and methods of the present invention with the advantages of a minimum of toxicity to the recipient cell or organism, with cellular access, intracellular trafficking and nuclear retention of plasmids.

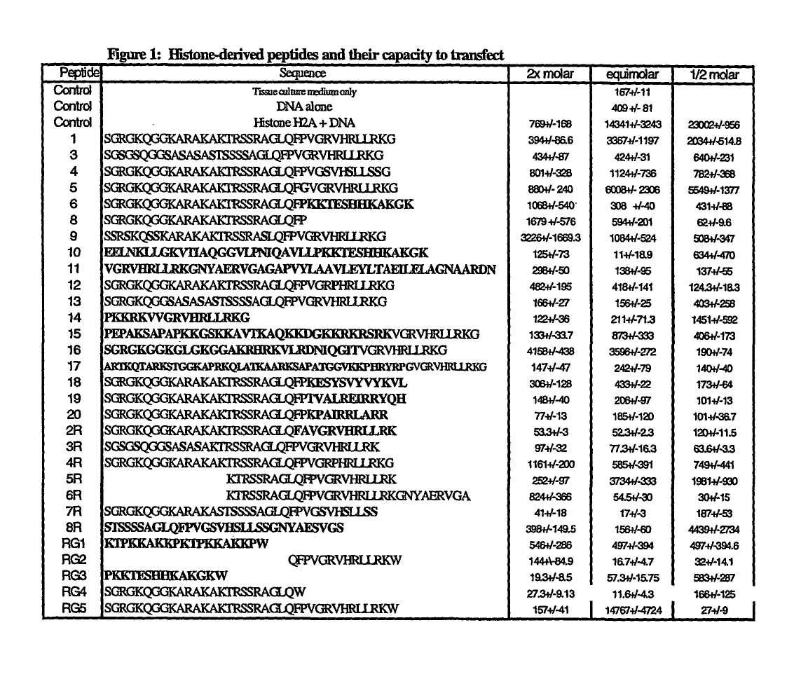

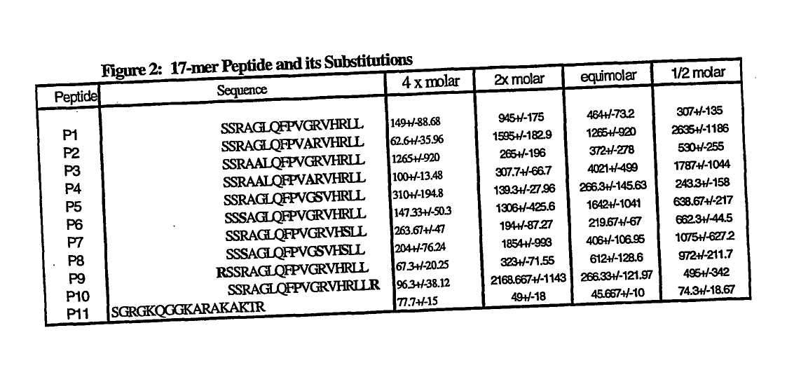

Thus, a gene delivery facilitating H2A-derived peptide of the present invention is a peptide of any length derived from the N-terminal domain or region of H2A that exhibits the ability to bind DNA. Preferably, a H2A-derived peptide further contains sufficient amino acid residues to mediate nuclear localization activity. The biologic properties of binding DNA and mediate nuclear localization are characteristics that are defined and as exemplified in the Examples. In preferred embodiments, the peptide minimally contains amino acid residues corresponding to amino acid residue positions 18-34 of native intact human H2A. A further preferred H2A-derived peptide contains amino acid residues corresponding to amino acid residue positions 1-36 or positions 1-37 of native intact human H2A. Amino acid substitutions, additions, deletions and the like permutations, including non-natural amino acids and D-form amino acids, which do not deleteriously effect the gene transfer function of the peptides are also contemplated in the context of peptide compositions in this invention. Homologous regions and permutations therein from other H2A species are also contemplated. Thus, the methods of this invention provide means to identify a gene delivery facilitating F12A-derived peptide having particular amino acid permutations of the N-terminal region. Particularly preferred peptides exhibiting the requisite activity of this invention are shown in FIGS. 1 and 2. In the context of the present invention, the term “delivery” is synonymous with “transfer” and the term “facilitate” is synonymous with “mediate”. The term “functional permutations” comprises such permutations of the peptides of the invention which retain transfection activity and nuclear localization activity.

In a preferred embodiment of the invention isolated gene delivery facilitating peptide comprises the amino acid motif knRnnRnnnnnnnnnRnnRnnRK, wherein n may be any amino acid. In a particular preferred embodiment the amino acid motif is nnRnKnnnKnRnKnKnRnnRnnnnnnnnRnnRnnRKn, wherein n may be any amino acid. Preferred amino acids which may take the positions designated “n” are the naturally occurring amino acids. Preferredly, the peptides of the invention retain transfection activity and nuclear localization activity.

Preferredly, the peptides have a transfection activity of at least twice background levels, preferably of at least three times background levels when measured in assays for transfection activity as described in the art. Preferredly, the assay as described in Example A, no. 1, is employed to measure transfection activity.

Nuclear localization activity within the meaning of the invention means any activity resulting in a rise in fluorescence in the cellular nucleus above background levels as may be assayed by methods known in the art, preferably by the method described in example 3A. Preferred is a rise in fluorescence above background levels of at least 10%, preferably at least 20%, at least 50% or even at least 2-fold.

Generally, the peptides of the invention may have any total length (total number of amino acids). Preferredly, they have a length of less than 129 amino acids. In particular, they may advantageously have a length of less than 50, or even less than 40 amino acids. In particular embodiments the peptides of the invention have a length of up to 37 amino acids, or they have a length of up to 17 amino acids. In preferred embodiments, they have a length of 37 amino acid, 36 amino acids or 17 amino acids. The peptides of the invention generally comprise at least 7 amino acids.

In another aspect the present invention provides an isolated complex comprising a histone H2A-derived peptide complexed with a nucleic acid. A H2A-derived peptide of this invention is effective at facilitating gene delivery without necessitating the use of additional reagents. Moreover, the H2A peptide-mediated delivery of a nucleic acid, such as an expression reporter gene plasmid, does not require the presence of cationic or anionic liposomes.

A gene delivery facilitating H2A-derived peptide is useful for the transfer of a nucleic acid including DNA and RNA. Preferred DNA molecules include 1) DNA from 5 nucleotides to 10,000 nucleotides in length, 2) DNA functionally ligated in an expression plasmid where the DNA can encode a cellular regulatory molecule, either activators or inhibitors, such as in naked DNA as more fully discussed below. Such molecules include tumor suppressor genes, genes that correct hereditary deficiencies, structural genes, and the like. Antisense nucleic acid molecules are also contemplated for use in the invention to inhibit the expression of undesirable genes. Preferred RNA molecules include mRNA for the expression of desired proteins. Mammalian, insect and bacterial nucleic acids are contemplated for use in the complexes and methods of this invention.

In preferred embodiments, a H2A-derived peptide of this invention is complexed with the nucleic acid in a transfection enhancing gram equivalent ratio of H2A-derived peptide:nucleic acid ranging from about 1/2.3 to 8000/1. A transfection enhancing gram equivalent ratio of H2A to nucleic acid, as taught in the Examples and in FIGS. 1 and 2, is the mass equivalent amount of H2A to complexed DNA that is efficiently transfected into a recipient cell. Preferred ratios include 1:2, 50:1, 100:1 and 200:1, 400:1, 6400:1, and 8000:1. Particularly preferred ratios for a shorter peptide of about 17 amino acid residues is 83:1 while the preferred ratio for a longer peptide of about 36 amino acid residues is 400:1. The range of peptide and DNA concentrations are further discussed in the Examples.

In another aspect of the invention, a H2A-derived peptide is useful as a recombinant protein expressed from an expression construct in which a DNA or RNA of interest is operatively linked to a nucleotide sequence encoding at least one gene delivery facilitating H2A-derived peptide. In one aspect, the peptide can be expressed as a fusion protein, although the invention contemplates a discistronic or multicistronic system in which the peptide is separately expressed for subsequent binding to the delivered nucleic acid to form a complex. Any construct is contemplated such that the binding of an expressed H2A-derived peptide or combination thereof is not hindered in the ability to mediate DNA binding. Thus, an expressed H2A-derived peptide or peptides can serve to facilitate the nuclear localization aspects of the peptides of this invention while conferring the advantages plasmid retention, lack of endosomal entrapment and protection from nucleases and the like cellular processes. In a further preferred embodiment, the H2A-derived peptide expression construct is further combined with a peptide of this invention to mediate efficient transfection capacity. The resultant complexes are preferably formed in the above stated ratios. Such a recombinant expression construct is exemplified by the one prepared for the nuclear localization assay as described more fully in the Examples.

In a further aspect of the H2A-derived peptide mediated gene delivery, the H2A-nucleic acid is present in a transfection enhancing medium that is defined as any medium in which the transfection efficiency of the complex is facilitated and not inhibited. A preferred transfection enhancing medium is Tris-acetate at a concentration between 1-125 mM at a pH 5.0 to pH 9.0. A particularly preferred transfection enhancing medium as further taught in the Examples is Tris-acetate medium is at 60 mM at pH 8.0. In the most preferred embodiment, the transfection enhancing medium lacks chloroquine and endotoxin.

Cells, either in vitro or in vivo, transfected with the H2A-derived peptide nucleic acid complexes as taught with the methods of the present invention are also contemplated. For in vitro embodiments, the cells include primary cultures of cells, cell lines, cells isolated from a subject for explant, i.e., ex vivo cultures, and the like. The use of the latter is described in U.S. Pat. No. 5,126,132, the disclosure of which is hereby incorporated by reference. Preferred cultured cells include mammalian, insect and bacterial cells. The present invention further contemplates the use of isolated stem cells of hematopoietic origin for use in diagnostic and therapeutic aspects of the invention. Isolation of such cells is described in U.S. Pat. Nos. 5,643,741 and 5,665,557, the disclosures of which are hereby incorporated by reference.

The invention also contemplates the use of transfecting a cell in vivo to mediate the efficient delivery of a desired nucleic acid to a cell. In such aspects, pharmaceutical compositions containing a transfection enhancing amount of a H2A-derived peptide nucleic acid complex is prepared in a pharmaceutically acceptable carrier. Candidate conditions for therapy include genetic diseases such as severe combined immunodeficiency, hemophilia A and B, familial hypercholesterolaemia, cystic fibrosis, hemoglobinopathies, Gaucher's disease, galactosemia, Tay Sachs, and include acquired diseases such as cancer, neurological diseases, cardiovascular conditions, and infectious diseases,

B. Methods of Making Gene Delivery Facilitating H2A-Derived Peptide Nucleic Acid Complexes

The invention provides methods for making a H2A-derived peptide nucleic acid complex. Exemplary methods of preparing a H2A-nucleic acid complex are described in the Examples where H2A-derived peptides are synthesized or expressed as recombinant proteins and combined with a solution of nucleic acid, such as plasmid DNA, prepared in a transfection enhancing medium A particularly preferred transfection enhancing medium is Tris-acetate as previously described.

C. Methods of Using Gene Delivery Facilitating H2A-Derived Peptide Nucleic Acid Complexes

Methods of using the H2A-derived peptide nucleic acid complexes on this invention are directed broadly to a method of transfecting a cell by administering a preferred complex to the cell. As previously discussed in Section A, the cell can either be in vitro or in vivo, with the noted preferred cells and applications thereof. In a further embodiment, transfection of a cell is accomplished with a H2A-derived peptide nucleic acid present in a transfection enhancing medium, also as previously discussed.

For in vitro embodiments, administering of a H2A-derived peptide nucleic acid complex comprises directly contacting the cultured cell with the complex. An exemplary method of contacting is described in the Examples where an aqueous solution of H2A-derived peptide nucleic acid complex is applied to a culture of cells in which the culture medium was immediately removed therefrom. In a preferred embodiment, the cultured cell is an ex vivo cultured cell. Contacting the cell with a H2A-nucleic acid complex results in the formation of a transfected ex vivo cell that can then be reintroduced into a compatible subject for delivering the desired nucleic acid to effect a desired outcome.

For in vivo embodiments as previously discussed in Section A and in the Examples, administering of a desired H2A-derived peptide nucleic acid complex, including an expression construct for expression of a H2A-derived peptide as previously discussed, can be accomplished by injection into a blood vessel, either arterial or venous, injection directly into a tumor, delivery by endoscopic means such as to bronchial airways and to colon, delivery intranasally, and the like. In the aspect for in vivo delivery, a pharmaceutical composition of a transfection enhancing amount of a H2A-derived peptide nucleic acid complex is provided in a pharmaceutically acceptable carrier, that is or can further contain a transfection enhancing medium as taught in the present invention.

1. Non-Viral Gene Transfer

Non-viral gene transfer as described in the present invention provides an alternative method of efficient gene delivery intended to result in lower levels of toxicity. The goal of non-viral gene therapy is mimicking the successful viral mechanisms for overcoming cellular barriers that block efficient expression of the target gene while minimizing the toxicities associated with gene delivery. The capabilities of a synthetic non-viral vector could include specific binding to the cell surface, entry, endosomal escape, translocation to the nucleus, and stable integration into the target cell genome. The rate limiting step of current non-viral gene delivery techniques is the transfer of encapsulated plasmids from the endosomes to the nucleus (Felgner, Sci. Am., 276:102-106, 1997). In this setting, plasmids are endocytosed by cells into the endosomal compartment. The acidity of this compartment together with its nuclease activity, would be expected to rapidly degrade plasmids (Felgner, Sci. Am., 276:102-106, 1997). Chloroquine is known to raise the acidic pH of endosomes, and is used in certain gene therapy protocols to promote endosomal release (Fritz et al., Hum. Gene Ther., 7:1395-1404, 1996).

The present invention provides for non-viral gene transfer that overcomes the inherent disadvantages associated with chemical and physical methods, including DEAE-dextran, polybrene and the mineral calcium phosphate, microinjection and electroporation. The present invention further is more useful than liposome gene transfer. While liposomal gene transfer has several advantages including lack of immunogenicity, ease of preparation, and the ability to package large DNA molecules, the ratio of liposome/DNA must be carefully controlled to circumvent the development of toxic aggregates. In addition, liposomes have a limited efficiency of delivery and gene expression, and they have potentially adverse interactions with negatively charged macromolecules.

2. H2A-Derived Peptide Mediated Gene Delivery

Complex formation with DNA in protein and peptide gene transfer, i.e., polyplex formation (Felgner et al., Hum. Gene Ther., 8:511-512, 1997), is mediated through electrostatic interactions between the positively charged lysine and arginine residues and the negatively charged phosphates in the DNA backbone (Sternberg et al., FEBS Lett., 356:361-366, 1994). Examples of peptide gene transfer exploit the physiological cellular process of receptor-mediated endocytosis for internalisation. Receptor-mediated gene delivery constructs contain a receptor-binding ligand and a DNA-binding moiety, usually poly-L-lysine. Cells have been targeted using a number of different ligands including transferrin, asialoglycoprotein, immunoglobulins, insulin, the EGF receptor, and an integrin binding-peptide. DNA binding elements include protamines, histones H1, H2A, H3 and H4, poly-L-lysine, and cationic amphiphilic α-helical oligopeptides with repeated sequences (Niidome et al., J. Biol. Chem., 272:15307-15312, 1997).

The potential advantages of protein/peptide gene transfer of the present invention include ease of use, production, and mutagenesis, purity, homogeneity, ability to target nucleic acids to specific cell types, cost effective large-scale manufacture, modular attachment of targeting ligands, and the lack of limitation on the size or type of the nucleic acid that can be delivered. The critical step for efficient gene delivery is the formation of the polyplex; analyses on the interactions between proteins/peptides and plasmids including particle size, protein/peptide/DNA charge ratio, buffering medium, and the like are underway to optimize the conditions for polyplex formation (Adami et al., J. Pharm. Sci., 87:678-683, 1998; Duguid et al., Biophys. J., 74:2802-2814, 1998; Murphy et al., Proc. Natl. Acad. Sci. USA, 95:1517-1522, 1998; Wadhwa et al., Bioconjug. Chem., 8:81-88, 1997). In contrast to the currently available methods of gene delivery which include calcium phosphate precipitation, DEAE dextran, electroporation, lipid systems, protein/peptide gene transfer involves the creation of a delivery vehicle whose properties can be predicted and controlled, and which could serve to enhance the activities required for the entry and persistent expression of exogenous nucleic acids. In addition, DNA condensation mediated via proteins/peptides stabilizes the polyplex during formulation and preserves its structure in serum, unlike many cationic liposomes (Adami et al, J. Pharm. Sci., 87:678-683, 1998; Wilke et al., Gene Ther., 3:1133-1142, 1996). Once active peptide motifs are identified, they can be combined to obtain a multifunctional complex with functions analogous to those of viral capsids. Over the last few years, a number of groups have become interested in protein/peptide gene transfer. However; in spite of the growing interest in this field, there is a paucity of information about the mechanism of action of protein/peptide-based vectors and discordant results regarding the effectiveness of this method of gene transfer. In addition, in vivo applications of protein/DNA polyplexes have been limited. The present invention describes compositions and methods of use that overcome these limitations.

3. Naked DNA

In the context of the present invention, naked DNA complexed with an H2A-derived peptide provides exemplary means to utilize efficient DNA expression in conjunction with peptide facilitated DNA binding, transfection and nuclear localization. Naked DNA, larger in size than oligonucleotides, is not readily endocytosed and must therefore be packaged into a vehicle capable of efficient entry into cells (Bongartz et al., Nucleic Acids Res., 22:4681-4688, 1994; Felgner, Sci. Am., 276:102-106, 1997). Naked DNA expression plasmids are described in U.S. Pat. Nos. 5,910,488, 5,693,622, 5,641,665 and 5,580,859, the disclosures of which are hereby incorporated by reference. The principal obstacle to cellular DNA uptake is charge (Felgner, Sci. Am., 276:102-106, 1997). In an aqueous solution, such as the milieu that bathes cells in the body, DNA has a net negative charge. DNA tends to be repelled from cell membranes, because they, too, are negatively charged. There are a few exceptions where cells appear to be able to assimilate naked DNA; this includes the successful target protein expression after direct muscular injections in mice (Blau and Springer, N. Engl. J. Med., 333:1554-1556, 1995; Cohen, Science, 259:1691-1692, 1993; Felgner, Sci. Am., 276:102-106, 1997; Wolff et al., Science, 247:1465-1468, 1990). While the mechanism of this type of gene transfer is unclear, a small amount of tissue damage or increased pressure at the injection site may play a role (Felgner, Sci. Am., 276:102-106, 1997). A few other types of cells and tissues can be transfected by the direct injection of naked DNA (Gao and Huang, Gene Ther., 2:710-722, 1995); these include the thyroid gland (Sikes et al., Gene Ther., 5:837-844, 1994), certain tumor types (Vile and Hart, Cancer Res., 53:962-967, 1993), and liver cells (Hickman et al., Hum. Gene Ther., 5:1477-1483, 1994). The remainder of the body is quite resistant to transfection unless a carrier is used.

D. Articles of Manufacture

A further aspect of this invention includes articles of manufacture containing a packaging material and a gene delivery facilitating H2A-derived peptide as described herein along with those shown in FIGS. 1 and 2 that is effective for transfecting a cell when complexed with nucleic acid. The packaging material contains a label or instructions for use which indicates that the H2A-derived peptide can be used along with how it is used for transfecting a cell with a nucleic acid when a H2A-derived peptide nucleic acid complex is formed. Additional components include anionic liposome, a lipid and an anionic polymer component. The article of manufacture is also prepared for use with a pharmaceutically H2A-derived peptide composition in a pharmaceutically acceptable carrier.

The following examples relating to this invention are illustrative and should not, of course, be construed as specifically limiting the invention. Moreover, such variations of the invention, now known or later developed, which would be within the purview of one skilled in the art are to be considered to fall within the scope of the present invention hereinafter claimed.

### EXAMPLES

A. Materials and Methods

1. Transfection Assay

The transfection assay is based on an in vitro assay previously described by J. H. Felgner et al. (Feigner et al., Proc. Natl. Acad. Sci., USA, 84:7413-7417, 1994) with the following modifications: COS-7 (African green monkey SV40-transformed kidney cells, American Type Culture Collection, Rockville, Md.) were maintained in Dulbecco's modified eagle's medium (Biowhittaker, Walkersville, Md.) supplemented with 10% heat inactivated fetal bovine serum (Gemini Bio-products Inc., Calabras, Calif.), 40 mM glutamine (Gemini Bio-products Inc., Calabras, Calif.), and 100U Penicillin-100 μg Streptomycin (Gemini Bio-products Inc., Calabras, Calif.). Other cells useful for transfection are 3T3, CHO-K1, HEPG2 and the like cell lines. The cells were harvested with 0.05% Trypsin-0.53 mM EDTA4Na (Life Technologies, Gaithersburg, Md.), pelleted, resuspended in their usual culture medium, diluted in 0.85% NaCl (Sigma, St. Louis, Mo.), and counted in a Coulter Z1® apparatus (Coulter Corporation, Miami, Fla.). COS-7 cells were plated in 96-well flat bottom tissue culture treated polystyrene plates (Corning Inc., Corning, N.Y.) at a density of 4×10⁵ cells per well and grown overnight in a humidified incubator at 37° C. in the presence of 4% CO₂. Culture medium was aspirated from the overnight cultures of COS-7 cells, and the cells were overlaid with 75 μl/well of the binary DNA-histone H2A-derived peptide complex, or the corresponding controls. Four hours after transfection, 37.5 μl of Opti-MEM®1, a Tris-Acetate-based (Life Technologies, Gaithersburg, Md.) containing 30% heat inactivated fetal bovine serum (Gemini Bio-products Inc., Celebres, Calif.) was added to each well. Twenty-four hours post-transfection 75 W of Opti-MEM®1 containing 10% heat inactivated fetal bovine serum (Gemini Bio-products Inc., Celebres, Calif.) was added to each well Forty-eight hours post-transfection, all of the medium in each well was removed. Fifty μl of lysis buffer (0.1% Triton X-100, 250 M Tris pH 8.0) was added to each well. The plates were then frozen at −70° C. and thawed once. Fifty μl of phosphate-buffered saline (PBS) pH 7.4 was added to each well except for the last column The last column was reserved for a β-galactosidase standard curve. In this column, 50 μl of two-fold serial dilutions of β-galactosidase grade VIII from E. coli (Sigma, St. Louis, Mo.) were made in PBS pH 7.4. Finally, 75 μl of 1.0 mg/ml chlorophenol red galactopyranoside (CPRG, Boehringer Mannheim, Indianopolis, Ind.) in β-galactosidase buffer (60 mM sodium dibasic phosphate pH 8.0, 1 mM magnesium sulfate, 10 mM KCl, 50 mM β-mercaptoethanol) were added in each well. In the presence of β-galactosidase, D-galactose is released from CPRG, yielding chlorophenol red; the red reaction product was used to quantitate the amount of β-galactosidase produced. The reaction was stopped by introducing 75 μl of 20% Tris base (pH 11) into each well after sufficient time had passed for the standard curve to be in the appropriate linear range, usually between 5-15 minutes after the introduction of CPRG. The plates were read in a Thermomax plate reader (Molecular Devices Incorporated, Sunnyvale, Calif.) at 575 nm. The optical density values obtained in test wells were then compared to those in the column containing the β-galactosidase standard and the result was expressed as the quantity of β-galactosidase produced per well. All assays were performed in triplicate. Complexes of histone H2A from calf thymus (Boehringer-Mannheim) with DNA were made using the most effective combinations for transfection that we previously described (Balicki and Beutler, supra, 1997).

Peptides were synthesized by Genemed Synthesis, Inc. (South San Francisco, Calif.) and occasionally by Research Genetics, Inc. Huntsville, Ala., and in the transfection described above. The plasmid DNA used throughout these experiments was pCMVβ (Clontech, Palo Alto, Calif.). pCWVβ is a β-galactosidase reporter plasmid under the control of a CMV promoter. Plasmid DNA was prepared using a Qiagen (Chatsworth, Calif.) Plasmid Giga kit and Endofree Plasmid Buffer Set. Dilutions of plasmid DNA were subjected to electrophoresis along with dilutions of Lambda DNA-Hind III Digest (New England Biolabs, Beverly, Mass.) on a 0.9% SeaKemGTG (Rockand, Me.) agarose gel in 1/2× Tris-phosphate (TPE) buffer. Circularized plasmid DNA was then quantitated using Stratagene's Eagle Eye II Still Video System (La Jolla, Calif.). Histone H2A, used as a control, was purchased from Boehringer Mannheim (Indianopolis, Ind.).

Whenever possible, large matrices of differing concentrations of DNA and peptide were assayed to determine the conditions for pea transfection results. In addition, a comparison of the ability of histone H2A-derived peptides to transfect COS-7 cells was made using equimolar ratios of peptides as compared with the active N-terminal 36mer of histone H2A. Also, a two-fold higher and two-fold lower molar ratio as were also tested for each peptide. Controls included tissue culture medium, DNA alone or DNA complexed with intact H2A. All samples were tested in triplicate. FIGS. 1 and 2 summarize these results displayed as the mean+/−standard error for each sample.

2. Confocal Microscope Analysis

Confocal studies were performed using a Zeiss Axiovert 100 fluorescent microscope attached to a laser and computer set-up utilizing BioRad's (Hercules, Calif.) MRC-1024 Confocal Laser Scanning System equipped with LaserSharp Software. Histone H2A was labeled with rhodamine while pCMVβ plasmid DNA was labeled with fluorescein isothiocyanate (FITC) using Panvera's Label IT™ kit (Madison, Wis.). The histone H2A-DNA complexes were prepared as above for the transfection of COS-7 cells with the modification that 10% of the histone H2A and plasmid DNA respectively was fluorescently labeled and that the cells were grown on coverglasses in Swell dishes. In addition, confocal microscopy was performed 24 hours after the start of transfection on cells washed four times with 1×PBS, and fixed with 4% paraformaldehyde (Electron Miscroscopy Sciences, Fort Washington, Pa.) for 10 min. at room temperature, followed by four more washes with 1×PBS. The fixed cells on coverglasses were subsequently mounted onto glass slides with a drop of Slowfade® (Molecular Probes, Eugene, Oreg.), and stored in the dark at 4° C. for future use.

3. Nuclear Localization

One type of functional assay to study the capability of a peptide to function as a nuclear localizing signal (NLS) is to fuse it to a protein that cannot, on its own, traffick into the nucleus; the β-galactosidase which is produced by the plasmid pCMVβ provides one such preferred exemplary model. The pCMVβ plasmid has a unique Xma I site at position 831-6. An Xma I site at position 967-972 of the nucleotide sequence of pCMVβ by mutagenizing the nucleotides “TC” to yield “GG” using a PCR-based method. The primers designed for this mutagenesis were:

GCTCAAGCGCGATCCCGGGGTTTTACAACGTCG and
CGACGTTGTAAAACCCCGGGATCGCGCTTGAGC.

The β-galactosidase gene of interest stretches from nucleotides 969-4013 in the pCMVβ plasmid. The result of the mutagenesis was the substitution of a glycine for a valine in β-galactosidase. The resultant plasmid was digested with Xma I, and a DNA fragment encoding either amino acids 1-36 or 18-34 of histone H2A was cloned into the Xma I sites; this DNA fragment was produced via a polymerase chain reaction using genomic DNA from a normal human donor as a template. This methodology results in the desired DNA sequence because histones are intronless. The sequences of the generated constructs were confirmed to correspond to the constructs of interest via automated DNA sequencing and double restriction digests using the enzymes Xho I and Fsp I.

The plasmids of interest were transformed into DH5α cells (Life Technologies, Gaithersburg, Md.). Plasmids were isolated using Qiagen's Plasmid Endofree kits (Qiagen, Chatsworth, Calif.) and used to transfect COS-7 cells grown on coverglasses in 6-well dishes using Superfect® (Qiagen) according to the manufacturer's recommendations. In alternative embodiments, a H2A-derived peptide is used instead of the Superfect® system to mediate transfection in conjunction with expressed peptide nuclear localization. Two days after the start of transfection, the cells were washed twice with PBS, and then subjected to indirect immunofluorescence as previously described (Neumann et al., J. Virol., 71:9690-9700, 1997), with the following modifications. First, the cells were fixed with 3.7% paraformaldehyde for 20 minutes at room temperature. Then, the cells were washed twice with PBS, incubated in the presence of ice cold acetone for 7 minutes at −20° C., and blocked with 10% goat serum (Sigma Immunochemicals, St. Louis, Mo.) for 20 minutes at 37° C. Subsequently, the cells were incubated overnight at 37° C. with a 1:200 dilution of monoclonal mouse anti-β-galactosidase antibody (Boehringer Mannheim, Indianopolis, Ind.). Thereafter, the cells were blocked with 10% goat serum (Sigma Immunochemicals, St. Louis, Mo.) for 20 minutes at 37° C., followed by an overnight incubation at 37° C. with a 1:200 dilution of FITC labeled goat anti-mouse IgG. The cells were washed with PBS, and visualized with a Zeiss Axiovert 100 inverted fluorescent microscope. Images were processed from a chip in a CCD camera attached to the microscope. Control experiments were performed with untransfected COS-7 cells, as well as COS-7 cells treated with DNA but with no Superfect®.

4. Circular Dichroism

Circular dichroism experiments were performed using a Circular Dichroism Spectrometer (model 62DS) at wavelengths ranging from 200-260 nm In all cases, three readings were taken of a 300 μl sample of 70 μM histone H2A or 70 μM histone H2A-derived peptide in 120 mM Tris acetate pH 8. The average reading for every wavelength tested is plotted as a function of degrees of ellipticity.

5. In Vivo Approaches

The present invention also contemplates use of the H2A-derived peptides to facilitate gene delivery in vivo. This embodiment is based on the successful subcutaneous immunization in a novel syngeneic mouse model of neuroblastoma with a single chain IL-12 fusion protein that was mediated by histone H2A transient transfection. The results indicated that the methods of this invention are effective to induce a T cell-mediated immunity that protects mice from challenge with wild type tumor cells as indicated by the complete absence of liver and bone metastases in 4/6 mice. In contrast, administration of immunization with IL-2 in this setting produced no tumor immunity. These results demonstrate the feasibility of transient transfection with histone H2A as well as the peptides of the present invention an efficient method of gene therapy with single chain IL-12 fusion protein, and provide the basis for application of this methodology to the clinical setting.

For this approach with intact histone H2A and also extendable to the peptides of this invention, mouse IL-2 and scIL-12 were cloned into the expression vector pcDNA3.1 (Invitrogen, Carlsbad, Calif.) as previously described (Lode et al., Proc. Natl. Acad. Sci., USA, 95:2475-2480, 1998). They were transformed into DH5α cells (Life Technologies, Gaithersburg, Md.), and plasmids were extracted using Qiagen's Endofree Plasmid kits and stored at −20° C. in LAL Reagent Water (Biowhittaker, Walkersville, Md.). Supercoiled DNA was quantitated on 0.9% agarose gels using Stratagene's Eagle Eye. NXS2 cells were plated in DMEM medium with 10% fetal calf serum (Hyclone, Utah), 100U/ml Penicillin-100 μg/ml Streptomycin (Life Technologies, Gaithersburg, Md.), and Glutamine (Life Technologies, Gaithersburg, Md.) at a density of 8×10⁵ cells per well of a 96 well plate, and were grown overnight at 37° C. and in a 5% humidified incubator. The following day, a 96-well matrix is prepared to optimize cytokine secretion with varying plasmid DNA and histone H2A (Boebringer Mannheim, Indianopolis, Ind.) or Superfect® (Qiagen, Chatsworth, Calif.) concentrations and used to overlay the NXS2 cells plated the day before. IL-12 and IL-12 production was quantified on a 24 hour basis in cell culture supernatants using an ELISA assay kit (Biosource, Camarillo, Calif.) and compared with standard preparations of these cytokines. Using the results from the 96-well matrices, smaller matrices were set up in 6-well plates.

NXS2 cells were tested routinely for the absence of mycoplasma contamination (Gen Probe® Mycoplasma Rapid Detection System, Fisher Scientific, Pittsburgh, Pa.). Actively growing NXS2 cells were plated at a density of 2.4×10⁷ cells per well of a 6 well plate, and were grown overnight at 37° C. and in a 5% humidified incubator. The following day, histone H2A was diluted in LAL Reagent Water (Biowhittaker, Walkersvile, Md.) and the expression vector was diluted in Tris Acetate pH 8 to a final concentration of 240 mM. In each well of a 6 well plate, 0.6 nl of Histone H2A were combined with 0.6 ml of plasmid DNA at room temperature for 30 minutes. Then, 1.2 ml of OptiMEM® (Life Technologies) medium were added to the histone-DNA mixture, resulting in a final Tris acetate concentration of 60 mM, pH 8. The medium of the overnight cultures was removed and replaced with the 2.4 ml mixture of histone-DNA and OptiMEM®. The cells were returned to grow at 37° C. in a 5% humidified incubator. Four hours later, 1.025 ml of OptiMEM® supplemented with 30% fetal calf serum was added to each well Twenty-four hours after the start of transfection, the overlying medium was aspirated and replaced with OptiMEM® supplemented with 10% fetal calf serum. Fourty-eight hours after the start of transfection, the medium was removed and stored at −70° C. until its use in an ELISA cytokine assay. Cells were harvested with 0.05% Trypsin-0.53 mM EDTA-4Na (Life Technologies, Gaithersburg, Md.). Cell viability was determined using trypan blue staining (Life Technologies, Gaithersburg, Md.), and the number of viable cells per experimental condition were determined. Cells were spun down and resuspended in unsupplemented DMEM medium for mouse injection.

Syngeneic female A/J mice were obtained at 6-8 weeks of age from The Jackson Laboratory or from a breeding colony at The Scripps Research Institute. Animal experiments were performed according to the National Institutes of Health Guide for The Care and Use of Laboratory Animals. Mice were first vaccinated subcutaneously in one abdominal flank with 2×10⁶ NXS2 cells determined by trypan blue staining to be at least 95% viable. Comparisons were made between equivalent numbers of cells transfected with histone H2A or Superfect® in the presence of the empty pcDNA3.1 vector and the pcDNA3.1 vector containing either the cDNA for mouse IL-2 or single chain IL-12. Primary tumor growth was determined over time by caliper measurements and the size calculated according to 1/2×width²×length. Seven to fourteen days later, experimental bone marrow and liver metastases were induced by tail vein injections of 5×10⁴ naive NXS2 cells. Control experiments were performed with mice receiving no prior vaccination. Mice were sacrificed for evaluation after 28 days. Liver weights were measured and the percentage of liver surface covered by fused metastases was determined. Bone marrow metastases were determined were evaluated by flushing the cavities of both femurs and tibiae of each mouse with 3 ml of PBS (pH 7.4). The resultant cell pellet was the source of total RNA isolation (RNeasy, Qiagen, Chatsworth, Calif.) and subsequent RT-PCR for the detection of tyrosine hydroxylase, as previously described (Lode et al., J. Natl. Cancer Institute, 89:1586-1594, 1997). High and low sensitvity PCR assays were performed. Bone marrow metastases were designated as stage 0 with no PCR signal stage 1 with an exclusive signal for high sensitivity PCR, and stage 2 in the presence of both high and low sensitivity PCR signals. Mechanistic studies were performed with specific antibody depletions prior to mouse vaccination and one week thereafter for NK cell depletion, and on a weekly basis for three consecutive weeks for T cell populations. Mice received intraperitoneal injections of anti-asialo-GM1 (Wako Bioproducts, Richmond, Va.), anti-CD4+, anti-CD8+ antibodies, or a combination of anti-CD4+ and CD8+ antibodies (Xiang et al., 1997). NK cells are depleted using the asialo-GMI antibody, while CD4+ and CD5+ T cell populations are depleted with anti-CD4+ and anti-CD8+ antibodies respectively.

H2A-derived peptides are separately prepared and used in the above approach for in vivo applications.

B. Results

1. Gene Delivery Facilitated With H2A-Derived Peptides

As shown in FIG. 1, a peptide corresponding to the first 36 amino acids of histone H2A was effective in delivering an expression plasmid into recipient cells, whereas peptides composed of amino acids 1-25, 26-36, and 116-129 of histone H2A are not, despite the high percentage of basic amino acid residues which may be helpful in electrostatic interactions with DNA. The difference between one of the effective peptides (amino acids 1-36) and the peptide composed of the first 25 amino acids is the inclusion of an α-helical motif located between amino acids 27-35. This result suggests that the secondary conformation of peptides may be related to their transfection activity. Subsequently, a 17-mer that represents amino acids 18-34 of the histone H2A molecule also was active in DNA delivery as shown in FIG. 2, albeit at a much higher molar ratio. X-gal staining revealed that histone H2A and the active 36-mer transfect between 5-10% of COS-7 cells, while the 17-mer transfects less than 1% of these cells.

Large transfection matrices were carried out with both the 36 and 17 mer to optimize their transfection of COS-7 cells. The peak activity for the 36-mer was observed with pCMVβ at a concentration of 20 μg/ml and with 15 μl of this peptide at 8 ng/ml Meanwhile, the 17-mer had an optimal transfection when the DNA was at a concentration of 120 μg/ml and when 6-12 μl of this peptide at 10 mg/ml was utilized in transfection.

Subsequently, a vast array of peptide substitutions of the 36-mer and the 17-mer were synthesized and compared. All these experiments were performed in triplicate on the same day, with the same batch of COS-7 cells. FIG. 1 charts the transfection activity of peptides at an equimolar ratio to the peak conditions for transfection of the 36-mer (peptide 1), as well as at a peptide concentration two-fold greater (2× molar) and two-fold less (½× molar). The results are the average β-galactosidase production +/− the standard error of the mean Peptide 5 has a glycine substitution at the start of an CL-helix; the flexibility of this amino acid may explain why this peptide has good levels of transfection. Peptide 6 combines the positively charged ends of the histone H2A molecule; transfection is low, once again suggesting that more than charge is operative in efficient transfection. Peptide 8 is from the N-terminus of peptide 1, displaying a slightly lower transfection ability. Peptide 10 is the C-terminal portion of histone H2A, displaying only background levels of transfection. Peptide 11 is from the middle of the histone H2A molecule, beginning at position 27; it too has background activity. Peptide 12 has a proline substituted for a valine in the first turn of an c-helix, which very interestingly suffices to bring transfection activity to background levels; once again emphasizing the important link between structure and function. Peptide 13 has multiple serine substitutions in the central part of the histone H2A molecule; transfection is low. Peptide 14 is the SV40 NLS and the C-terminus of peptide 1; some activity is detectable. Peptides 15, 16, and 17 have the α-helix of histone H2A as their C-terminus, and the N-terminal region of histone H2B, H4, and H3, respectively. Peptides 18, 19, and 20 have the N-terminal region of histone H2A, and the first α-helical region of histones H2B, H3, and H4 respectively. Peptides 18, 19 and 20 have the N-terminal region of histone H2A, and the first α-helical region of histones H2B, H3 and H4, respectively.

Peptide 16 has significant transfection activity, possibly emanating from a favorable conformational change generated by its N-terminus. Peptide 2R has an alanine substituted for a proline, that may nucleate the α-helix of peptide 1. Once again the transfection activity drops significantly suggesting that this substitution is also critical. Peptide 3R has multiple serines instead of arginines and lysines, emphasizing the importance of charge in transfection. Peptide 4R is identical to peptide 12, some loss of transfection activity is seen. Peptide 5R is a 22 amino acid component of peptide one that also contains the active 17-mer. It has significant activity under the same conditions as peptide 1's peak activity. These structural studies suggest that this region contains most of the DNA-binding sites of histone H2A; they are depicted here in bold font:

KTRSSRAGLQFPVGRVHRIIRK (peptide 5R)

Peptide 6R extends peptide 5R, with a loss of transfection activity. Peptide 7R resembles histone H2A, but has some serine substitutions; its activity is significantly decreased. Peptides 8R and RG1 are molecules given to us by a collaborator. Peptide RG2 contains an α-helix of histone H2A deemed to be important; on its own it has little activity. RG4 has the N-terminus of peptide 1. RG5 is the full-length active peptide, with a transfection activity comparable to the whole histone H2A molecule. P1 is the 17-mer, that has only background activity when tested at equimolar concentrations to those used for peptide 1. However, when used at a different optimal concentration, P1 was effective in DNA transfection and delivery. FIG. 2 summarizes the transfection activity of some 17-mers that are identical to P1 except for a few substitutions. Peptide P11 is a short stretch of amino acids found in the N-terminus of Peptide 1. Glycines or arginines are substituted in the remaining peptides. An additional arginine was also added at either end of peptide P1. Some increased transfection activity occurs when the N-terminal arginine of peptide P1 is substituted by serine, possibly by freeing this end.

2. Confocal Analysis

The preliminary data from confocal microscopic studies indicated that histone H2A serves to localize the complexes to the nucleus. In these studies, plasmid DNA was covalently labeled with fluorescein isothiocyanate (FITC), while histone H2A was labeled with rhodamine. Complexes of FITC-labeled DNA and rhodamine-labeled histone H2A were then formed by mixing these components together with unlabeled DNA and histone H2A. COS-7 cells were then transfected by these labeled complexes. Confocal microscopy was chosen for these experiments as it provides fluorescent visualization of thin sections of cellular compartments at high resolution. Rhodamine-labeled and FITC-labeled particles of the same slice of cells can be analyzed individually. These analyses were then merged, corrected for bleed through, and then analyzed for colocalization of fluorescent labels. After a 24 hour transfection of COS-7 cells with fluorescently labeled particles, both rhodamine and FITC signals were found to colocalize in the nucleus. Control experiments using transfection of COS-7 cells with FITC-labeled DNA in the absence of histone H2A showed no nuclear localization of the fluorescently labeled DNA. These data indicated that the histone H2A-DNA complex entered the nucleus.

3. Nuclear Localization Analysis

β-galactosidase is a proven test system for deciphering nuclear localization signals (Moreland et al., Mol. Cell Biol., 7:4048-4057, 1987; Neumann et al., J. Virol., 71:9690-9700, 1997). β-galactosidase is a cytoplasmically localized protein, but fusion with the nuclear localization signals of the influenza virus nucleoprotein and a basic region in the C terminus of the retinoblastoma gene product 110RB1 cause it to go to the nucleus. To examine whether the H2A-derived peptides of the present invention had nuclear localization signal properties, a construct (pCMVβ-NLS) was made wherein the nucleotide sequence corresponding to the active 36-mer was cloned immediately upstream of the β-galactosidase coding region of the reporter plasmid pCMVβ. COS-7 cells were transfected using a commercial transfecting reagent, Superfect®, and with the reporter plasmid pCMVβ or the pCMVβ-NLS plasmids. β-galactosidase was expressed for 48 hours after transfection.

4. Circular Dichroism Analysis

Circular dichroism of peptide was performed on four samples: the full length histone H2A molecule, peptide 1 (36-mer), peptide P1 (17-mer) and peptide 12. Peptide 12 is identical to peptide 1, except that it has a proline in its first α-helical turn; it also has decreased activity in transfection that may be related to its structure. The circular dichroism of histone H2A is compatible with α-helical structure, which are classically represented by minima at 208 and 222 nm wavelength. Both peptide 1 and P1 have some degree of minima at around 208. Interestingly, peptide 12 has no evidence of α-helical structure.

### C. CONCLUSION

There is a correlation between structure and function in gene delivery facilitating histone H2A-derived peptide-mediated transfection. Structural studies suggest that histone H2A has a unique organization with a clip of DNA-binding sites clustered in its N-terminus. Peptides derived from the N-terminal region of histone H2A were shown to efficiently mediate DNA binding, transfection and nuclear localization, thereby functioning as competent gene delivery facilitating peptides. Substitutions of amino acids of this peptide reveal that electrostatic interactions, DNA binding sites, and structural organization (e.g. secondary structure) are key for effective transfection. The latter was evidenced by disruption of the α-helix of peptide 12, and the subsequent decline of this peptide's transfection activity.

Other variations and uses of the present invention will be apparent to one skilled in the art in light of the present disclosures.

What is claimed is:

1) An isolated gene delivery facilitating peptide comprising at least 7 amino acids, preferredly 17 amino acids, derived from the N-terminal region of histone H2A, wherein the peptide exhibits transfection activity and nuclear localization activity.

2) The peptide of claim 1 wherein the peptide does not have the sequence of the full-length wild type human H2A protein.

3) The peptide of claim 1 or 2 comprising the amino acid sequence SSRAGLQFPVGRVHRLL, and functional permutations thereof.

4) The peptide of any of claims 1-3 comprising the amino acid sequence SGRGKQGGKARAKAKTRSSRAG LQFPVGRVHRLLRKG, and functional permutations thereof.

5) An isolated gene delivery facilitating peptide comprising the amino acid motif KnnRnnRnnnnnnnnnRnnRnnRK, wherein n may be any amino acid.

6) The peptide of claim 5 comprising the amino acid motif nnRnKnnnKnRnKnKnRnnRnnnnnnnnRnnRnnRKn, wherein n may be any amino acid.

7) The peptide of any of claims 1 to 6 having a transfection activity of at least twice background levels, preferably of at least three times background levels when measured in the assay as described in Example A, number 1.

8) A complex comprising a peptide according to any of claims 1-7 complexed with a nucleic acid.

9) The complex of claim 8 wherein the nucleic acid is an antisense molecule.

10) The complex of claim 8 wherein the nucleic acid is an expression plasmid.

11) The complex of claim 10 wherein the expression plasmid encodes a reporter molecule.

12) The complex of claim 10 wherein the expression plasmid encodes at least one gene delivery facilitating H2A-derived peptide according to any of claims 1-7.

13) The complex of claim 10 wherein the expression plasmid encodes a regulatory molecule.

14) The complex of claim 13 wherein the regulatory molecule is a cellular inhibitor or a cellular activator.

15) The complex of any of claims 8-14 wherein the peptide is complexed with the nucleic acid in a transfection enhancing gram equivalent ratio of peptide: nucleic acid ranging from 1:2.3 to 8000:1.

16) A solution comprising the complex of any of claims 8-15 and a transfection enhancing medium.

17) The solution of claim 16 wherein the transfection enhancing medium comprises Tris-acetate.

18) The solution of claim 17 wherein the Tris-acetate medium is between pH 5.0 to pH 9.0, preferredly about pH 8.0.

19) The solution of claim 17 wherein the Tris-acetate medium is between 1-125 mM, preferredly about 60 mM.

20) The solution of any of claims 16-19 wherein the solution lacks chloroquine and endotoxin.

21) A pharmaceutical composition comprising a transfection enhancing amount of a complex according to any of claims 8-15 in a pharmaceutically acceptable carrier.

22) A method of preparing a complex comprising mixing a peptide according to any of claims 1-7 with a nucleic acid in a transfection enhancing medium to form a peptide nucleic acid complex.

23) The method of claim 22 wherein the transfection enhancing medium comprises Tris-acetate.

24) The method of claim 23 wherein the Tris-acetate medium is between pH 5.0 to pH 9.0, preferredly about pH 8.0.

25) The method of claim 23 wherein the Tris-acetate medium is between 1-125 mM, preferredly about 60 mM.

26) The method of any of claims 22-25 wherein the transfection enhancing medium lacks chloroquine and endotoxin.

27) A method of transfecting a cell comprising administering a complex according to any of claims 8-15 to the cell.

28) The method of claim 27 wherein the cell is a cultured cell.

29) The method of claims 27 or 28 wherein the cultured cell is selected from the group consisting of mammalian, insect and bacterial cells.

30) The method of claim 28 wherein the cultured cell is an ex vivo culture.

31) The method of claim 30 wherein the ex vivo culture comprises stem cells.

32) The method of claim 27 wherein the cell is in vivo.

33) The method of any of claims 27-32 wherein the complex is present in a solution according to any of claims 16-20.

34) The method of claim 28 wherein administering comprises directly contacting the cultured cell with the complex.

35) The method of claim 30 wherein administering comprises directly contacting the ex vivo cultured cell with the complex to form a transfected ex vivo cell.

36) The method of claim 30 wherein the transfected ex vivo cell is reintroduced to a compatible subject.

37) The method of claim 34 wherein administering comprises injection into a blood vessel, injection into a tumor, delivery by endoscopic means, and delivery intranasally.

38) A cell transfected according to a method of any of claims 27-37.

39) An article of manufacture comprising a packaging material and contained therein in a separate container a gene delivery facilitating H2A-derived peptide according to any of claims 1-7, wherein the peptide is effective for delivering a nucleic acid into a cell, and wherein the packaging material comprises a label which indicates that the peptide can be used for delivering a nucleic acid into a cell when a H2A-derived peptide nucleic acid complex is formed.

40) An article of manufacture comprising a packaging material and contained therein in a separate container a pharmaceutical composition comprising a gene delivery facilitating H2A-derived peptide according to any of claims 1-7, in a pharmaceutically acceptable carrier, wherein the peptide is effective for delivering a nucleic acid into a cell, and wherein the packaging material comprises a label which indicates that the peptide can be used for delivering a nucleic acid into a cell when a H2A-derived peptide nucleic acid complex is formed.

41) A nucleic acid coding for a peptide of any of claims 1-7.


SEQUENCE LISTING (46 sequences)

SEQ ID NO: 1 - protein, 129 aa, homo sapiens
SGRGKQGGKARAKAKTRSSRAGLQFPVGRVHRLLRKGNYAERVGAGAPVYLAAVLEYLTAEILELAGNAARDNKKTRIIPRHLQLAIRNDEELNKLLGKVTIAQGGVLPNIQAVLLPKKTESHHKAKGK

SEQ ID NO: 2 - protein, 22 aa, Artificial Sequence
  generic amino acid motif
KXRXXRXXXXXXXXRXXRXXRK

SEQ ID NO: 3 - protein, 37 aa, Artificial Sequence
  generic amino acid motif
XXRXKXXXKXRXKXKXRXXRXXXXXXXXRXXRXXRKX

SEQ ID NO: 4 - DNA, 33 bp, Artificial Sequence
  synthetic primer
gctcaagcgcgatcccggggttttacaacgtcg

SEQ ID NO: 5 - DNA, 33 bp, Artificial Sequence
  synthetic primer
cgacgttgtaaaaccccgggatcgcgcttgagc

SEQ ID NO: 6 - protein, 37 aa, Artificial Sequence
  synthetic histone-derived peptide
SGRGKQGGKARAKAKTRSSRAGLQFPVGRVHRLLRKG

SEQ ID NO: 7 - protein, 37 aa, Artificial Sequence
  synthetic histone-derived peptide
SGSGSQGGSASASASTSSSSAGLQFPVGRVHRLLRKG

SEQ ID NO: 8 - protein, 37 aa, Artificial Sequence
  synthetic histone-derived peptide
SGRGKQGGKARAKAKTRSSRAGLQFPVGSVHSLLSSG

SEQ ID NO: 9 - protein, 37 aa, Artificial Sequence
  synthetic histone-derived peptide
SGRGKQGGKARAKAKTRSSRAGLQFGVGRVHRLLRKG

SEQ ID NO: 10 - protein, 38 aa, Artificial Sequence
  synthetic histone-derived peptide
SGRGKQGGKARAKAKTRSSRAGLQFPKKTESHHKAKGK

SEQ ID NO: 11 - protein, 26 aa, Artificial Sequence
  synthetic histone-derived peptide
SGRGKQGGKARAKAKTRSSRAGLQFP

SEQ ID NO: 12 - protein, 37 aa, Artificial Sequence
  synthetic histone-derived peptide
SSRSKQSSKARAKAKTRSSRASLQFPVGRVHRLLRKG

SEQ ID NO: 13 - protein, 39 aa, Artificial Sequence
  synthetic histone-derived peptide
EELNKLLGKVTIAQGGVLPNIQAVLLPKKTESHHKAKGK

SEQ ID NO: 14 - protein, 47 aa, Artificial Sequence
  synthetic histone-derived peptide
VGRVHRLLRKGNYAERVGAGAPVYLAAVLEYLTAEILELAGNAARDN

SEQ ID NO: 15 - protein, 37 aa, Artificial Sequence
  synthetic histone-derived peptide
SGRGKQGGKARAKAKTRSSRAGLQFPVGRPHRLLRKG

SEQ ID NO: 16 - protein, 37 aa, Artificial Sequence
  synthetic histone-derived peptide
SGRGKQGGSASASASTSSSSAGLQFPVGRVHRLLRKG

SEQ ID NO: 17 - protein, 17 aa, Artificial Sequence
  synthetic histone-derived peptide
PKKRKVVGRVHRLLRKG

SEQ ID NO: 18 - protein, 45 aa, Artificial Sequence
  synthetic histone-derived peptide
PEPAKSAPAPKKGSKKAVTKAQKKDGKKRKRSRKVGRVHRLLRKG

SEQ ID NO: 19 - protein, 41 aa, Artificial Sequence
  synthetic histone-derived peptide
SGRGKGGKGLGKGGAKRHRKVLRDNIQGITVGRVHRLLRKG

SEQ ID NO: 20 - protein, 55 aa, Artificial Sequence
  synthetic histone-derived peptide
ARTKQTARKSTGGKAPRKQLATKAARKSAPATGGVKKPHRYRPGVGRVHRLLRKG

SEQ ID NO: 21 - protein, 38 aa, Artificial Sequence
  synthetic histone-derived peptide
SGRGKQGGKARAKAKTRSSRAGLQFPKESYSVYVYKVL

SEQ ID NO: 22 - protein, 38 aa, Artificial Sequence
  synthetic histone-derived peptide
SGRGKQGGKARAKAKTRSSRAGLQFPTVALREIRRYQH

SEQ ID NO: 23 - protein, 36 aa, Artificial Sequence
  synthetic histone-derived peptide
SGRGKQGGKARAKAKTRSSRAGLQFPKPAIRRLARR

SEQ ID NO: 24 - protein, 36 aa, Artificial Sequence
  synthetic histone-derived peptide
SGRGKQGGKARAKAKTRSSRAGLQFAVGRVHRLLRK

SEQ ID NO: 25 - protein, 36 aa, Artificial Sequence
  synthetic histone-derived peptide
SGSGSQGGSASASAKTRSSRAGLQFPVGRVHRLLRK

SEQ ID NO: 26 - protein, 37 aa, Artificial Sequence; identical to SEQ ID NO: 15
  synthetic histone-derived peptide

SEQ ID NO: 27 - protein, 22 aa, Artificial Sequence
  synthetic histone-derived peptide
KTRSSRAGLQFPVGRVHRLLRK

SEQ ID NO: 28 - protein, 31 aa, Artificial Sequence
  synthetic histone-derived peptide
KTRSSRAGLQFPVGRVHRLLRKGNYAERVGA

SEQ ID NO: 29 - protein, 36 aa, Artificial Sequence
  synthetic histone-derived peptide
SGRGKQGGKARAKASTSSSSAGLQFPVGSVHSLLSS

SEQ ID NO: 30 - protein, 31 aa, Artificial Sequence
  synthetic histone-derived peptide
STSSSSAGLQFPVGSVHSLLSSGNYAESVGS

SEQ ID NO: 31 - protein, 19 aa, Artificial Sequence
  synthetic histone-derived peptide
KTPKKAKKPKTPKKAKKPW

SEQ ID NO: 32 - protein, 14 aa, Artificial Sequence
  synthetic histone-derived peptide
QFPVGRVHRLLRKW

SEQ ID NO: 33 - protein, 14 aa, Artificial Sequence
  synthetic histone-derived peptide
PKKTESHHKAKGKW

SEQ ID NO: 34 - protein, 25 aa, Artificial Sequence
  synthetic histone-derived peptide
SGRGKQGGKARAKAKTRSSRAGLQW

SEQ ID NO: 35 - protein, 37 aa, Artificial Sequence
  synthetic histone-derived peptide
SGRGKQGGKARAKAKTRSSRAGLQFPVGRVHRLLRKW

SEQ ID NO: 36 - protein, 17 aa, Artificial Sequence
  synthetic histone-derived peptide
SSRAGLQFPVGRVHRLL

SEQ ID NO: 37 - protein, 17 aa, Artificial Sequence
  synthetic histone-derived peptide
SSRAGLQFPVARVHRLL

SEQ ID NO: 38 - protein, 17 aa, Artificial Sequence
  synthetic histone-derived peptide
SSRAALQFPVGRVHRLL

SEQ ID NO: 39 - protein, 17 aa, Artificial Sequence
  synthetic histone-derived peptide
SSRAALQFPVARVHRLL

SEQ ID NO: 40 - protein, 17 aa, Artificial Sequence
  synthetic histone-derived peptide
SSRAGLQFPVGSVHRLL

SEQ ID NO: 41 - protein, 17 aa, Artificial Sequence
  synthetic histone-derived peptide
SSSAGLQFPVGRVHRLL

SEQ ID NO: 42 - protein, 17 aa, Artificial Sequence
  synthetic histone-derived peptide
SSRAGLQFPVGRVHSLL

SEQ ID NO: 43 - protein, 17 aa, Artificial Sequence
  synthetic histone-derived peptide
SSSAGLQFPVGSVHSLL

SEQ ID NO: 44 - protein, 18 aa, Artificial Sequence
  synthetic histone-derived peptide
RSSRAGLQFPVGRVHRLL

SEQ ID NO: 45 - protein, 18 aa, Artificial Sequence
  synthetic histone-derived peptide
SSRAGLQFPVGRVHRLLR

SEQ ID NO: 46 - protein, 17 aa, Artificial Sequence
  synthetic histone-derived peptide
SGRGKQGGKARAKAKTR